(19) United States
(12) Patent Application Publication
(10) Pub. No.: US 2026/0224209 A1
(43) Pub. Date: Aug. 6, 2026

(54) MALLEABLE RETRACTOR WITH INTEGRATED SUCTION

(71) Applicants: University of Pittsburgh - Of the Commonwealth System of Higher Education, Pittsburgh, PA (US); UPMC, Pittsburgh, PA (US)

(72) Inventors: Michael McDowell, Hummelstown, PA (US); Ricardo Fernandez-deThomas, Guaynabo, PR (US); Benjamin Leslie, Export, PA (US); Christopher Holliday, Pittsburgh, PA (US)

(21) Appl. No.: 19/150,584

(22) PCT Filed: Jan. 26, 2024
(86) PCT No.: PCT/US2024/013096

§ 371 Date: Jul. 24, 2025

Related U.S. Application Data
(60) Provisional application No. 63/441,346, filed on Jan. 26, 2023.

(51) Int. Cl. A61B 17/02; A61B 17/00

(52) U.S. Cl. CPC A61B 17/02; A61B 2017/00946; A61B 2217/005

(57) ABSTRACT

A retractor includes an extending malleable body and a suction system in connection with the extending malleable body. At least a portion of the suction system is configured to conform to a conformation of the extending malleable body while maintaining suction functionality. The suction system includes at least one conduit via which material from a site can pass through the suction system. The suction system is configured to be placed in fluid connection with a vacuum source.

### CROSS-REFERENCE TO RELATED APPLICATIONS

This application claims benefit of U.S. Provisional Patent Application Ser. No. 63/441,346, filed Jan. 26, 2023, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Malleable retractors, sometimes referred to a ribbon retractors, are used for retraction (holding away) of tissue in various parts of the body (for example, in connection with surgery of the brain, spine, intestines, other organs, etc.) to provide access to a region of interest. Malleable retractors are typically made from malleable, medical-grade metals (for example, stainless steel). The malleability of the retractors allows the retractors to be shaped to varying conformations according to the area in which the surgery is being performed, but with sufficient rigidity to be able to adequately retract soft tissues without change in conformation. Multiple designs of the sterilizable, lightweight, and flexible ribbon retractors are available and can be used in various types of surgeries.

Both suction and manual retraction are very important for visualization in, for example, intracranial, intraspinal, and visceral surgeries. However, there is very limited space in such surgeries which is occupied by an increasing number of different instruments.

### SUMMARY

In one aspect, a retractor or surgical retractor includes an extending malleable body and a suction system in connection with the extending malleable body. At least a portion of the suction system is configured to conform to a conformation of the extending malleable body while maintaining suction functionality. The suction system includes at least one conduit via which material from a site (for example, a surgical site) can pass through the suction system. The suction system is configured to be placed in fluid connection with a vacuum source (that is, a source of negative pressure or suction).

In a number of embodiments, the extending malleable body includes an interior volume. The interior volume may, for example, be in fluid connection with one or more passages formed in a wall of the extending malleable body. In a number of embodiments, the interior volume forms a part of the at least one conduit.

The suction system in a number of embodiments includes a suction pathway element which is flexible and is configured to pass through at least a portion of the internal volume. The flexible suction pathway element includes the at least one conduit and at least one port in fluid connection with the at least one conduit. The at least one port is in fluid connection with the one or more passages formed in a wall of the extending malleable body.

The suction pathway element may, for example, be formed from a flexible polymeric material. In a number of embodiments, the suction pathway element is formed from a silicone material or another biocompatible polymeric material.

In a number of embodiments, the extending malleable body includes a first section and the second section that are configured to be placed in an open state and in a closed state, wherein the first section and the second section form the interior volume when in a closed state. At least one of the first section or the second section may include the one or more passages.

The suction system may, for example, include a suction pathway element which is flexible and insertable into the internal volume when the first section and the second section are in an open state to pass through at least a portion of the internal volume. The suction pathway element includes the at least one conduit and at least one port in fluid connection with the at least one conduit. The at least one port is in fluid connection with the one or more passages formed in a wall of the extending malleable body. In a number of embodiments, the suction pathway element forms a sealed engagement with the interior volume to create a suction chamber when the first section and the second section are in a closed state, the suction chamber being in fluid connection with the one or more passages, and the at least one port being positioned within the suction chamber.

The extending malleable body may be formed from a malleable metal. In a number of embodiments, the malleable metal is medical-grade stainless steel or other medical-grade metal or metal alloy.

In a number of embodiments, the suction system includes a suction pathway element which is flexible and is configured to be connected to the extending malleable body. The flexible suction pathway element forms at least a portion of the exterior surface of the surgical retractor. The flexible suction pathway element includes the at least one conduit and at least one port in fluid connection with the at least one conduit.

The suction pathway element is formed from a flexible polymeric material. In a number of embodiments, the suction pathway element is formed from a silicone material or another biocompatible polymeric material.

The extending malleable body may, for example, include an extending section to which the suction pathway element is attachable. The suction pathway element may include a seating into which a portion of the extending malleable body may be positioned to attached the extending section to the suction pathway element.

In a number of embodiments, the extending section includes a suction chamber positioned on a distal end thereof. The suction chamber includes an opening on a proximal end thereof via which suction volume positioned on a distal end of the suction pathway element can be passed into the suction chamber. The suction chamber includes one or more passages therein. The at least one port is positioned in the suction volume to be placed in fluid connection with the one or more passages when the distal end of the suction pathway element is within the suction chamber.

The suction pathway element may form at least a portion of an upper surface and a lower surface of the surgical retractor in a number of embodiments. The seating may extend the full length of the suction pathway element. In a number of embodiments, the suction pathway element includes a generally flat upper surface, a generally flat lower surface and a curved surface connecting the generally flat upper surface and the generally flat lower surface. A distal end of the suction pathway element may include a rounded edge.

In a number of embodiments, the extending section of the malleable body is U-shaped, and the suction pathway element includes a U-shaped slot around a circumference thereof in which the extending section is seated when the suction pathway element is attached to the extending section. The suction pathway element may, for example, include a generally flat upper surface, a generally flat lower surface and a curved surface connecting the generally flat upper surface and the generally flat lower surface. A distal end of the suction pathway element may include a rounded edge.

In another aspect, a method of performing a procedure includes using a retractor during the procedure. The retractor includes an extending malleable body and a suction system in connection with the extending malleable body. At least a portion of the suction system is configured to conform to a conformation of the malleable body while maintaining suction functionality. The suction system includes at least one conduit via which material from a site can pass through the suction system. The suction system is configured to be placed in fluid connection with a vacuum source. The procedure may, for example, be a surgical procedure. However, the retractors herein may be used in connection with any procedure in which retraction/displacement and suction are desirable.

The method may further include bending the extending malleable body of the retractor to a (desired) conformation. Bending the extending malleable body of the retractor may be achieved manually. Bending the extending malleable body of the retractor may be achieved using a bending tool.

The method may further include retaining the retractor in a position during the procedure by operatively connecting the retractor to a retention system. The retention system may, for example, be connected to a robotic control system.

As described above, in a number of embodiments, the extending malleable body includes an interior volume. The interior volume may, for example, be in fluid connection with one or more passages formed in a wall of the extending malleable body. In a number of embodiments, the interior volume forms a part of the at least one conduit.

The suction system in a number of embodiments includes a suction pathway element which is flexible and is configured to pass through at least a portion of the internal volume. The flexible suction pathway element includes the at least one conduit and at least one port in fluid connection with the at least one conduit. The at least one port is in fluid connection with the one or more passages formed in a wall of the extending malleable body.

The suction pathway element may, for example, be formed from a flexible polymeric material. In a number of embodiments, the suction pathway element is formed from a silicone material or another biocompatible polymeric material.

In a number of embodiments, the extending malleable body includes a first section and the second section that are configured to be placed in an open state and in a closed state, wherein the first section and the second section form the interior volume when in a closed state. At least one of the first section or the second section may include the one or more passages.

The suction system may, for example, include a suction pathway element which is flexible and insertable into the internal volume when the first section and the second section are in an open state to pass through at least a portion of the internal volume. The suction pathway element includes the at least one conduit and at least one port in fluid connection with the at least one conduit. The at least one port is in fluid connection with the one or more passages formed in a wall of the extending malleable body. In a number of embodiments, the suction pathway element forms a sealed engagement with the interior volume to create a suction chamber when the first section and the second section are in a closed state, the suction chamber being in fluid connection with the one or more passages, and the at least one port being positioned within the suction chamber.

The extending malleable body may be formed from a malleable metal. In a number of embodiments, the malleable metal is medical-grade stainless steel or other medical-grade metal or metal alloy.

In a number of embodiments, the suction system includes a suction pathway element which is flexible and is configured to be connected to the extending malleable body. The flexible suction pathway element forms at least a portion of the exterior surface of the retractor. The flexible suction pathway element includes the at least one conduit and at least one port in fluid connection with the at least one conduit.

The suction pathway element is formed from a flexible polymeric material. In a number of embodiments, the suction pathway element is formed from a silicone material or another biocompatible polymeric material.

The extending malleable body may, for example, include an extending section to which the suction pathway element is attachable. The suction pathway element may include a seating into which a portion of the extending malleable body may be positioned to attached the extending section to the suction pathway element.

In a number of embodiments, the extending section includes a suction chamber positioned on a distal end thereof. The suction chamber includes an opening on a proximal end thereof via which suction volume positioned on a distal end of the suction pathway element can be passed into the suction chamber. The suction chamber includes one or more passages therein. The at least one port is positioned in the suction volume to be placed in fluid connection with the one or more passages when the distal end of the suction pathway element is within the suction chamber.

The suction pathway element may form at least a portion of an upper surface and a lower surface of the retractor in a number of embodiments. The seating may extend the full length of the suction pathway element. In a number of embodiments, the suction pathway element includes a generally flat upper surface, a generally flat lower surface and a curved surface connecting the generally flat upper surface and the generally flat lower surface. A distal end of the suction pathway element may include a rounded edge.

In a number of embodiments, the extending section of the malleable body is U-shaped, and the suction pathway element includes a U-shaped slot around a circumference thereof in which the extending section is seated when the suction pathway element is attached to the extending section. The suction pathway element may, for example, include a generally flat upper surface, a generally flat lower surface and a curved surface connecting the generally flat upper surface and the generally flat lower surface. A distal end of the suction pathway element may include a rounded edge.

In another aspect, a kit or system includes (i) one or more malleable bodies and a plurality of suction pathway elements connectable to each of the one or more malleable bodies as described herein or (ii) a plurality of malleable bodies and at least one suction pathway element connectable to each of the plurality of malleable bodies. In a number of embodiments, the kit or system includes a plurality of malleable bodies and a plurality of suction pathway elements connectable to each of the plurality of malleable bodies. Each of the one or more malleable bodies and each of the one or more suction pathway elements may be different to provide different combinations of malleable bodies and suction pathway elements in a particular retractor.

The present devices, systems, and methods along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a top plan view of embodiments of malleable retractors hereof with integrated suction functionality.

FIG. 1B illustrates a top plan view of further embodiments of malleable retractors hereof with integrated suction functionality.

FIG. 1C illustrates a top plan view of further embodiments of malleable retractors hereof with integrated suction functionality.

FIG. 1D illustrates a top plan view of further embodiments of malleable retractors hereof with integrated suction functionality.

FIG. 1E illustrates a top plan view of further embodiment of malleable retractors hereof with integrated suction functionality.

FIG. 2A illustrates a top plan views of first and second sections of another embodiment of malleable retractor hereof including one or more channels or conduits of differing shape, width, length etc. formed in the first section and one or more openings of different shape, width, length etc. formed in the second section.

FIG. 2B illustrates a top plan view of the first and second sections of another embodiment of malleable retractor hereof.

FIG. 2C illustrates a top plan view of the first and second sections of another embodiment of malleable retractor hereof.

FIG. 2D illustrates a top plan view of the first and second sections of another embodiment of malleable retractor hereof.

FIG. 3 illustrates a side view of an embodiment of a malleable retractor hereof including a release mechanism for the first and second sections of the malleable retractor that may be used to detach or open the first and second sections for various functions or operations.

FIG. 4 illustrates several side views of the malleable retractor of FIG. 3 demonstrating the functionality of the malleable retractor to preserve suction functionality after being bent at different locations along the entire length of the malleable retractor, including bends of different magnitude as well as multiple bends at different radii or angles.

FIG. 5 illustrates a top plan view of another embodiment of a malleable retractor hereof in a disconnected state in which a removable suction pathway element or chamber is configured to be placed within a volume or space formed between the first section and the second section of the malleable retractor upon attachment thereof, wherein the first section and the second section may, for example, be detached or opened for cleaning, disposal and/or replacement of the suction pathway element.

FIG. 6A illustrates an isometric view of another embodiment of a malleable retractor hereof.

FIG. 6B illustrates an isometric, view of the malleable retractor of FIG. 6A with the first and second section of a malleable body of the malleable retractor in an open state and a pathway suction element removed from the malleable body.

FIG. 7A illustrates an isometric view of another embodiment of a malleable retractor hereof.

FIG. 7B illustrates an isometric, view of the malleable retractor of FIG. 7A wherein a suction pathway element thereof is disconnected from a malleable body or frame member thereof.

FIG. 8A illustrates an isometric view of another embodiment of a malleable retractor hereof including malleable body and a flexible suction path element which is removably connectible to the malleable body.

FIG. 8B illustrates an isometric, view of the malleable retractor of FIG. 8A in an exploded or disconnected state wherein the flexible suction path element thereof is disconnected from a malleable body and a vacuum or suction line connector is disconnected from the suction path element.

FIG. 8C illustrates an isometric, hidden line view of the malleable retractor of FIG. 8A wherein the suction path element is removably connected to the malleable body.

FIG. 9A illustrates a top plan, schematic view of a malleable retractor as illustrated in FIG. 1A in operative connection with a connector of a retaining system.

FIG. 9B illustrates a side, schematic view of the malleable retractor in operative connection with a connector of the retaining system of FIG. 9A, wherein the malleable retractor is oriented toward the viewer.

FIG. 10A illustrates a side, schematic view of bending of the extending malleable body of a malleable retractor hereof with an embodiment of a bending tool, wherein the upper view illustrates the malleable retractor placed in operative connection with the bending tool, and the lower view illustrates bending of the malleable retractor after application of force to handles of the bending tool.

FIG. 10B illustrates a side, schematic view of bending of the extending malleable body of a malleable retractor hereof with another embodiment of a bending tool hereof.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “conduit” includes a plurality of such conduit and equivalents thereof known to those skilled in the art, and so forth, and reference to “the conduit” is a reference to one or more such conduits and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

Suction may, for example, be used to clear the field of obstructing fluids, debris, and/or other obstructions. As described above, although manual retraction is very valuable for, for example, visualization in surgeries (for example, in intracranial, intraspinal, and visceral surgeries), there is very limited space in many such surgeries. Moreover, many instruments may need to share that limited space. One such instrument is a medical suction instrument, sometimes referred to simply as suction.

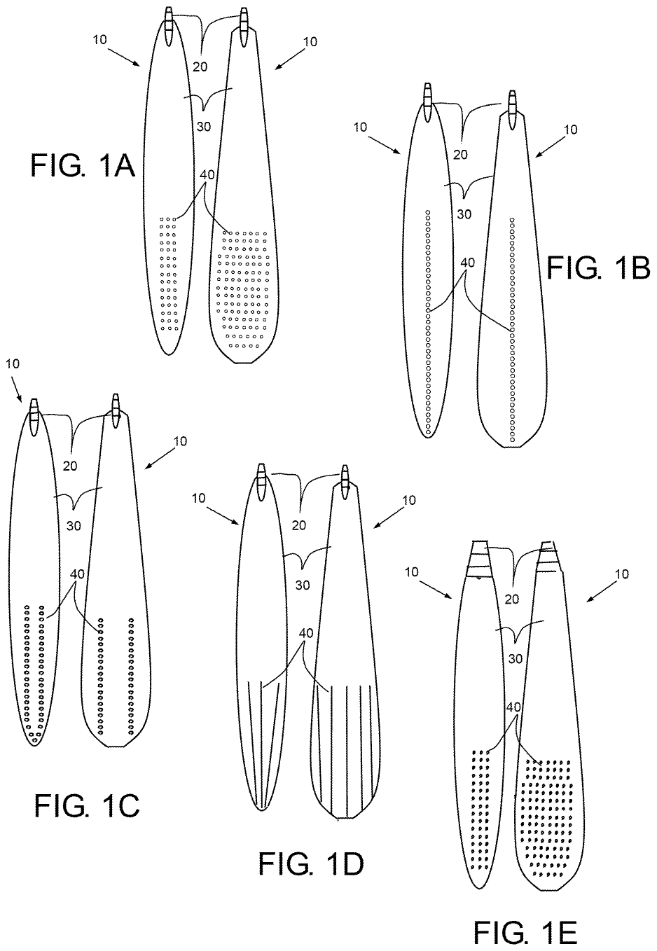

In a number of embodiments hereof, suction functionality is combined or integrated into a malleable retractor. Such retractors may be ribbon retractors which are instruments with a flexible, ribbon-like extending section or blades. Smaller ribbon retractors may, for example, be used in pediatric procedures and in neurosurgical or ophthalmic procedures. Larger ribbon retractors may, for example, be used in abdominal or cardiovascular procedures (for example, to hold back larger organs). Combination of such devices provides improved visualization while providing continuous suction. FIGS. 1A through 1E illustrates several embodiments of malleable retractors hereof with integrated suction. Referring to, for example, FIG. 1A, in each such embodiment of a malleable retractor 10, an attachment or line attachment 20 is provided to connect to a source of low/negative pressure, suction or vacuum. Line attachment 20 may, for example, include a barbed or other fluid line/tubing connector as known in the art. Line attachment 20 is in operative connection with an internal space or volume of a malleable body 30 which forms or is operable as a suction system as described further below. In the embodiments of FIG. 1A through 1E, one or more passages, openings or entry points 40 is/are provided over the surface of malleable body 30 in fluid connection with the internal volume of malleable body 30, and thereby with line attachment 20. As illustrated FIG. 1A through 1E, the size and shape of malleable body 30 is variable over a wide range. Likewise, passages 40 may vary in number, size, shape, distribution, etc. over a wide range thereof.

Malleable retractors 10 and/or malleable body 30 may, for example, be formed (at least in part) from a malleable metal such as a medical or surgical grade stainless steel or other metal (for example, iron/metal alloys). Such devices can also be made, at least in part, of non-metals like silicone or coated with nonconducting materials such as silicone to avoid electric current conduction during cautery. It is desirable for malleable body 30 to be formed from a malleable material. Such materials, may for example, be nonbrittle, have minimal reactivity or inertness on contact with human tissues, be noncorrosive, be nonporous, be nonflammable, be thrombosis resistance (for the time of exposure), be heat resistant (for example, for multi-use embodiment which undergo sterilization), and be nontoxic. With respect to the malleability of such a material or materials, the material(s) should be sufficiently rigid to resist the forces experienced during application to tissue during a surgical procedure but sufficiently malleable or shapeable to be able to be manipulated (that is, bent, folded, twisted etc.) manually or with a bending instrument. Properties such as nonconductivity and resistance to abrasion (from example, from exposure to a drill) are properties useful in various embodiment, depending upon use. A choice of metal or a coating therefor may, for example, be used to achieve such properties. In a number of embodiments, malleable retractors hereof (or at least portions thereof such as, for example, metal portions thereof) are suitable for cleaning, sterilization, and reuse. In other embodiments, malleable retractors (or at least portions thereof) may be designed for a single use before disposal. For single-use devices, a variety of polymers (such as, polyurethane, polypropylene), resins (such as epoxy, acrylic, polyester), and plastics may be used.

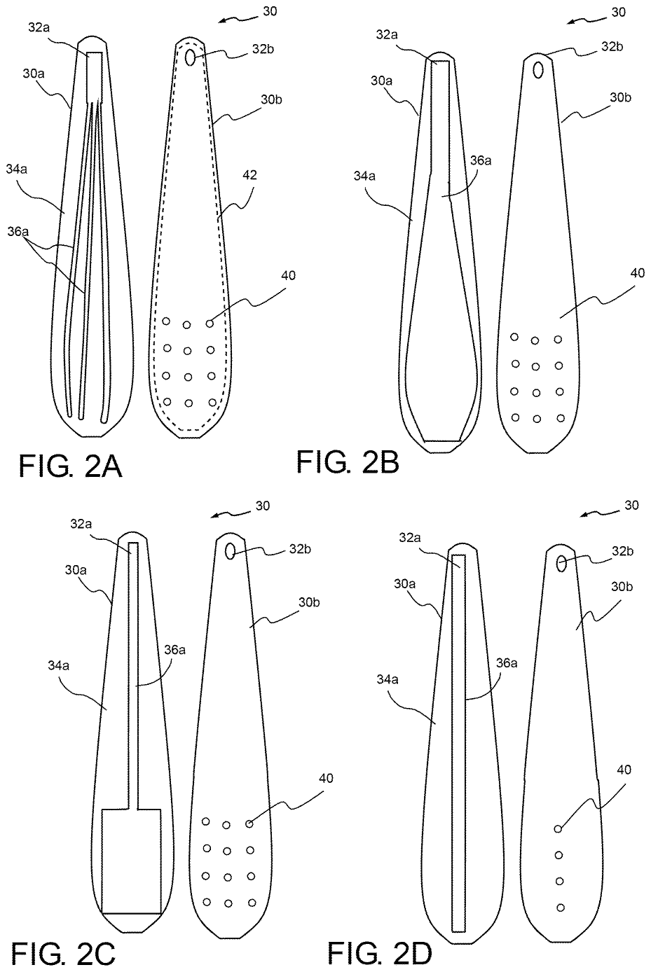

FIGS. 2A through 2D illustrate several embodiments of malleable body 30 (without line attachment 20 attached thereto) wherein a first section 30a and a second section 30b of malleable body 30 are illustrated in an open or separated/disconnected state. As illustrated in FIGS. 2A through 2D, second section 30b includes an attachment port 32b which cooperates with line attachment 20 to connect to a source of low pressure. When first section 30a and second section 30b are placed in a closed or connected state, port 32b is placed in fluid connection with a portal section 32a formed in first section 30a (for example, as a recess, cavity, or volume in an interior surface 34a of first section 30a) which forms part of the internal volume of malleable body 30. Portal section 32a may, for example, operate in the manner of a manifold to receive flow from one or more conduits or channels 36a (which may be formed as recesses in interior surface 34a) which extend through first section 30a to be placed in fluid connection with passages 40. Second section 30b may include similar recesses (not shown in FIGS. 2A through 2D) to form the internal volume of malleable body 30 upon connection of first section 30a and second section 30b. Such extending conduits 36a may, for example, vary significantly in shape, width, length, distribution etc. to be placed in fluid connection with one or more openings 40 (which may vary in number, size, shape, distribution, etc. as described above). In the embodiments of FIGS. 2A through 2D attachment port 32b is positioned in second section 30b. However, in other embodiments such an attachment port may be formed in the top (in the orientation illustrated in FIGS. 1A through 2D) of one or both of first and second sections 30a and 30b (see, for example, FIG. 1E) to a cooperate with a suction line attachment 20 positioned at the top end of malleable body 30. It may desirable, but not necessary, to position or orient such all or most of such ports so that they are located at positions other than (for example, on an opposite side of the retractor from) the side or sections thereof used to contact and retract tissue. One or more sealing members 42 (illustrated schematically in dashed lines in FIG. 2A) as known in the mechanical sealing arts (such as a section or layer of an elastomeric material) may be provided in or one of both of first and second sections 30a and 30b to ensure a sealed connection therebetween in embodiments in which malleable body 30 is formed to have two openable or disconnectable sections.

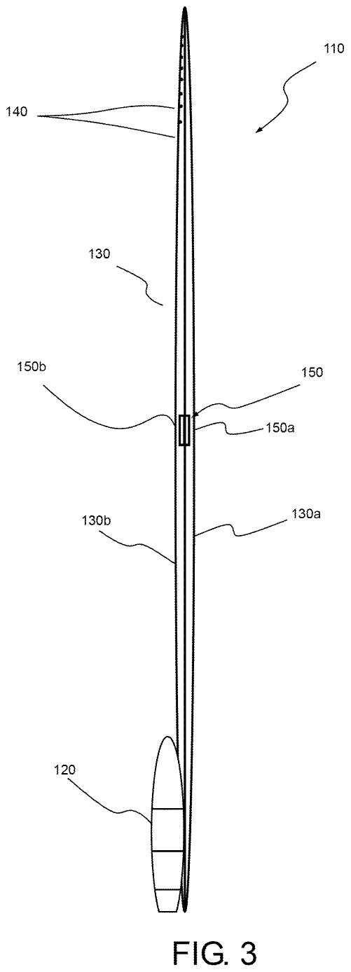

FIG. 3 illustrates a side view of a malleable retractor 110 hereof including one or more closure/release mechanisms 150 to control opening and closing of a first section 130a and a second section 130b of a malleable body 130. Mechanism(s) 150 include a first connector 150a attached to first section 130a and a second or cooperating connector 150b attached to second section 130b. Second connector 150b includes passages, openings or entry points 140. Release mechanism(s) 150 may, for example, be used to latch/detach first section 130a and second section 130b of malleable body 130 which may for example, open in a manner similar to a clam shell via a hinged connection (not shown in FIG. 3) or may be completely separable for completion of various functions or operations.

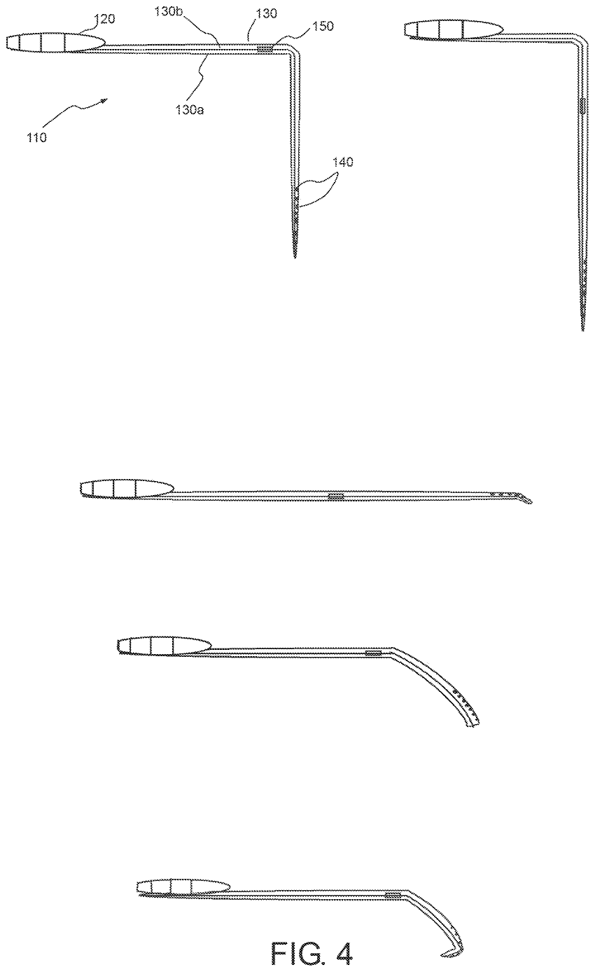

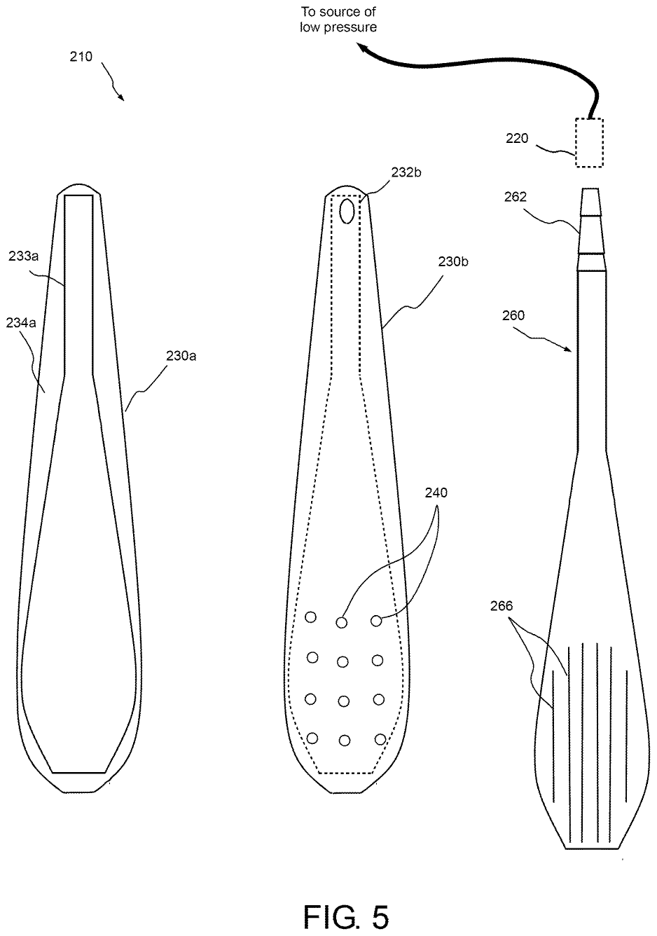

FIG. 4 illustrates the functionality of malleable body 130 of malleable retractor 110 of FIG. 3 to be bent or formed to any number of different conformations (based on surgical retraction needs) while preserving the suction functionality thereof. Malleable body 130 may be bent at one or more different locations along the length and/or width of malleable body 130. Such bends may be curved or curvilinear and have arcs/angles of different radii or degree and different length/magnitude.

FIG. 5 illustrates another embodiment of a malleable retractor 210 hereof in which a suction system hereof includes a removable suction pathway, conduit or chamber element 260, which is configured to be placed within an interior volume or space 233a formed by a recess in the inner surface of one or both of the first section 230a and the second section 230b of malleable body 230 upon attachment thereof. Suction pathway element 260 provided an efficient system to convey low/negative pressure, suction, or vacuum. Suction pathway element 260 may, for example, be formed from a flexible polymeric material (for example, a flexible polymeric materials such as a silicone material) which is suitable for use as a fluid pathway element to deliver/apply suction as described further below.

Pressure typically applied in surgical suction is, for example, approximately 520 mm Hg (negative gauge pressure) or lower. The materials of suction pathway elements hereof have a sufficient modulus to provide adequate compression/tension when the suction pathway element is bent into various configurations, and such that the material channels do not collapse as a result of applied negative pressure. In a number of embodiments, materials (for example, polymeric materials) for used in suction pathway elements hereof have a modulus in the range of 1-200 GPa.

In general, the desirable material properties set forth above for the materials of the malleable bodies hereof also apply to the materials for the suction pathway elements hereof (other than heat resistance because such suction pathway elements will typically be single-use components). In general, any property inherent to a metal or other materials for the malleable body would also be applicable to the suction pathway element hereof. Typically, if a material can be used for the suction pathway element, it can be used for the malleable body and vice versa. However, differences may arise depending upon the use count that material provides (that is, one use vs indefinitely reusable via sterilization). Polymeric materials suitable for use in suction pathway element hereof include, for example, silicon, polycarbonate, polyethylene polypropylene, and nylon. In a number of studied embodiments hereof high density polyethylene or AGILUS™ (a rubber-like, photopolymer available from Stratasys of Eden Prairie, Minnesota US) was used in suction pathway elements.

In general, a number of polymers such as silicon and other polymers discussed above inherently provide a significant degree of hemocompatibility. Moreover, surface coatings may be applied to increase hemocompatibility for silicon and other polymers/materials. For example, grafting or ionically attaching heparin has been shown to reduce thrombosis. Polyurethane has, for example, been modified with 2-methacryloyloxyethyl phosphorylcholine (MPC) which provided good anticoagulation. Metal-phenolic catecholamine coatings, hyaluronic/polydopamine coatings, and ultrananocrystalline diamond coatings can also provide improved blood compatibility. Coatings are, for example, discussed in Wilson A C, Neuenschwander P F, Chou S F. Engineering Approaches to Prevent Blood Clotting from Medical Implants. Arch Biomed Eng Biotechnol; 1(2) (2019), the disclosure of which is incorporated herein by reference

In the illustrated embodiment of FIG. 5, suction pathway element 260 includes a connector section 262 designed to pass through an opening or passage 232b in section 230b to connect to or form a part of a suction line attachment 220 (illustrated schematically in broken lines FIG. 5). Suction pathway element 260 also includes ports or passages 266 (extending, linear passages or slots in the embodiment illustrated in FIG. 5) formed in a surface or wall thereof. Matter from outside of malleable retractor 210 is drawn in through holes or passages 240 and intro ports 266 via the suction transmitted through suction pathway element 260. First section 230a and second section 230b can be detached or opened for cleaning, disposal thereof and/or replacement of chamber 300.

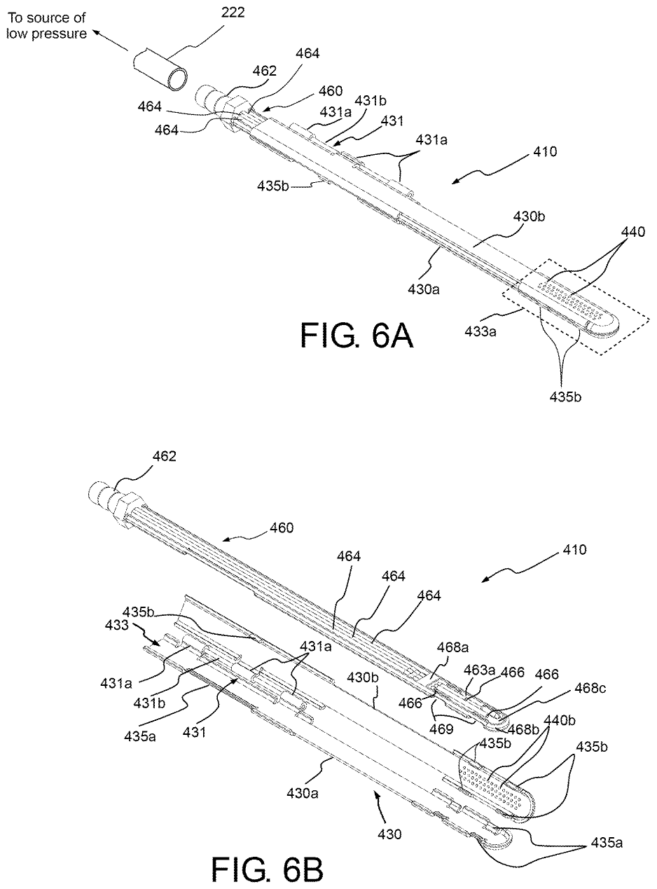

FIGS. 6A and 6B illustrates another embodiment of a malleable retractor 410 hereof including a removable suction pathway element 460 configured to be placed or positioned within an interior volume or space 433 formed by the inner surfaces of first section 430a and the second section 430b of a malleable body 430 upon attachment/closure thereof. As described in connection with suction pathway element 260, suction pathway element 460 may, for example, be formed from a flexible polymeric material such a silicone material.

In the illustrated embodiment of FIGS. 6A and 6B, first section 430a and the second section 430b are connected via a hinging connector 431. In the illustrated embodiments, hinging connector 431 includes elements 431a which cooperate with an extending pin element 431b. Elements 431a and pin element 431b provide relative sliding motion to allow for stress relief in the case of bending. In that regard, bending may affect first section 430a and second section 430b differently in terms of length change. First section 430a further includes one or more connectors 435a and second section 430b includes one or more cooperating connectors 435b which cooperate via flexing or snaping connection or interaction to place body 430 in a closed state (see FIG. 6A) and in an open state (FIG. 6B).

Suction pathway element 460 includes a connector section 462 including, for example, a barbed tubing connector or attachment to a suction line tubing 222 (see, FIG. 6A; for example, of a hospital suction system). In the illustrated embodiment, suction pathway element 460 also includes one or more extending flexible conduits 464 (three in the illustrated embodiment) which include ports or opening 466 positioned within a suction space or volume 463a in a proximal end of suction pathway element 460. In the illustrated embodiment, openings are positioned on the distal end of flexible conduits 464 but may additional or alternatively be positioned on the sides, bottoms, and/or tops of flexible conduits 464. Surfaces of suction pathway element 460 (for example, surfaces 468a and 468b as well as bottom surface 468c) may, for example, form a sealing or substantially sealing engagement with inner surfaces of malleable body 430 at a distal end thereof and form a suction chamber 433a (encompassed in broken lines in FIG. 6A). In the illustrated embodiment, surface 468b includes surface features such as recesses 469 which cooperate with and/or conform to features of at least one of first section 430a and second section 430b (for example, connectors 435a and/or cooperating connectors 435b) to retain suction pathway element 460 in operative connection with malleable body 430 when first section 430a and second section 430b are in a closed state.

Openings 440 formed in second section 430b are in fluid connection with suction chamber 463a. As further illustrated in FIG. 6B, the position of ports 466 may be staggered to distribute low pressure/suction within volume 433a and suction chamber 463a formed thereby. Matter from outside of malleable retractor is drawn in through holes or passages 440 and intro ports 466 to pass through flexible conduits 464. As described above in connection with malleable body 230 of malleable retractor 210, first section 230a and second section 230b can be detached or opened for cleaning, or for disposal and/or replacement of suction pathway element 430.

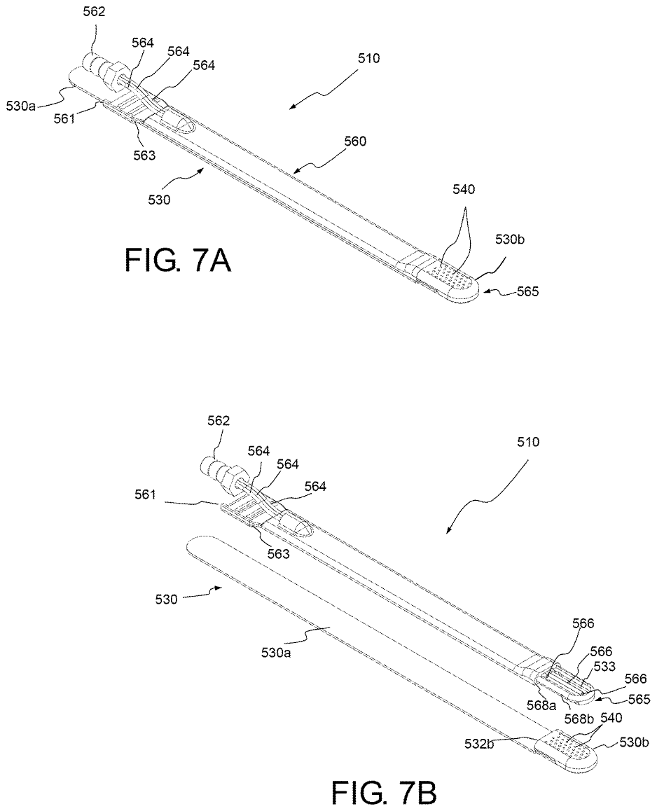

FIGS. 7A and 7B illustrates another embodiment of a malleable retractor 510 hereof including a suction system including a removable suction pathway element 560 configured to be placed in connection with a malleable body 530 thereof. In the illustrated embodiment of FIGS. 7A and 7B, malleable body 530 includes an extending section 530a and a chamber section 430b attached to or formed upon a distal end of extending section 530a. Malleable body 530 may, for example, before formed as an integral or monolithic mass of material (for example, a malleable metal as known in the metal arts). As described further below, suction pathway element 560 extends over a top (in the illustrated orientation) surface of malleable body 530 and forms part of or a portion of the outside surface of malleable retractor 510 when connected to extending section 530a thereof.

Suction pathway element 560 may, for example, be formed from a flexible polymeric material such a silicone material as described above. Suction pathway element 560 includes or is attached to a connector element or connector 562 such as a barbed tubing connector or attachment to connect to a suction line tubing 222 (see, FIG. 6A). Suction pathway element 560 also includes one or more extending flexible conduits 564 (three in the illustrated embodiment) which are in fluid connection with connector element 562 at a proximal end thereof and include ports or openings 566 at a distal end thereof within a suction space of volume 533. In the illustrated embodiment of FIGS. 7A and 7B, suction pathway element 560 includes a proximally positioned connecting section 561 including one or more slots or seatings 563 through which extending section 530a can be slid relative to suction pathway element 560 during connection thereof to suction pathway element 560 or removal thereof from suction pathway element 560.

During connection of suction pathway element 560 to malleable body 430, extending section 530a may be slid in a rearward or proximal direction relative to suction pathway element 560 so that the distal end thereof passes through and forms a connection with connectors such as slot(s) 563 and a distal section 565 of suction pathway element 560 (including suction volume 533) passes into an opening 532b of chamber section 530b. Additional connectors such as slots may be provided along the length of suction pathway element 560 for interaction with or connection to malleable body 530. A single, continuous connector such as a slot may be provide over the length of malleable body 530. In a number of embodiments, malleable body 530 and suction pathways element 560 are held together by friction. One or more cooperating abutment element may be provided to so assist in holding malleable body 530 and suction pathways element 560 in operative connection at a desired relative position. Surfaces of distal section 565 of suction pathway element 560 (for example, surfaces 568a and 568b) may form a sealing engagement with surfaces of chamber section 530b, forming a sealed suction chamber within chamber section 530b (see FIG. 7B). Upon such connection, openings 540 formed in chamber section 530b are placed in fluid connection with suction volume 533. As described above in connection with openings or ports 466, the position of ports 566 may be staggered to distribute low pressure/suction within the suction chamber formed by chamber section 530b and suction volume 533. During use of malleable retractor 510, matter from outside of malleable retractor is drawn in through holes or passages 540 and into ports 566 while other sections (including, the opposing side and sides) of malleable retractor 510 contact and retract tissue.

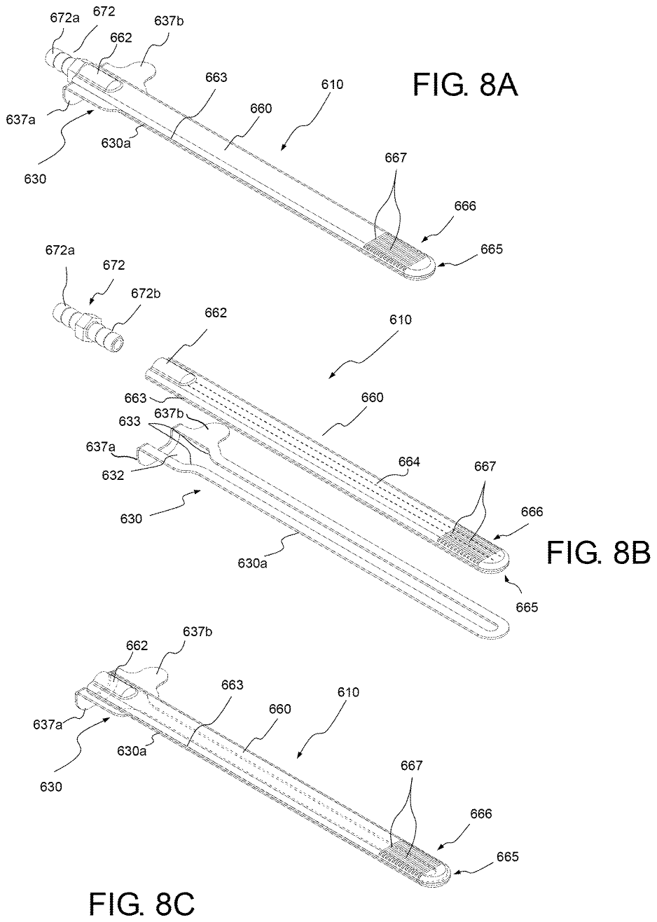

FIGS. 8A through 8C illustrates another embodiment of a malleable retractor 610 hereof including a removable suction pathway element 660 configured to be placed in connection with a malleable body 630 of the malleable retractor 610. In the illustrated embodiment of FIGS. 8A through 8C, malleable body 630 includes a generally U-shaped extending section or frame 630a. Such an extending section or frame includes two, spaced axially extending members which may or may not be connected at a distal end thereof. In the illustrated embodiment of FIGS. 8A through 8C, the extending members are connected via an arced distal section, forming a U-shape.

As described above, suction pathway element 660 may, for example, be formed from a flexible polymeric material such a silicone material. In the illustrated embodiment, suction pathway element 660 includes a connector section 672 which is configured to connect to a fluid connector such as a barbed tubing connector 672 to connect to a suction line tubing 222 (see, FIG. 6A). In the illustrated embodiment, connector 672 includes a first barbed section 672a for connection to suction line tubing 222 and a second barbed section 672b for connection to a connector section 662 of suction pathway element 660 to place suction pathway element 660 in fluid connection with source of suction, low/negative pressure or vacuum (for example, a vacuum pump of a hospital system). Connector section 672a may alternatively be formed as a portion of suction pathway element 660.

Suction pathway element 660 further includes a generally U-shaped slot or seating 663 into which at least a portion of extending section or member 630a may be positioned during removable connection of suction pathway element 660 and malleable body 630. In that regard, during connection of suction pathway element 660 to malleable body 630, distal end 665 of suction pathway element 660 may be passed through an opening or gap 632 on a proximal end of extending section 630a so that U-shaped slot 663 can contact and seat the axially extending members of extending section 630a therein. Sections 633 of opening or gap 632 may be arced or curved to extend inward toward a centerline of extending section 630a to facilitate engagement of slot 663 in the vicinity of distal end 665. Once extending section 630a is initially engaged within slot 663 in the vicinity of sections 633, suction pathway element 660 is further advanced or slid forward relative to the axially extending member of extending section 630a of malleable body 630 until extending section 630a is fully seated withing slot 663 as illustrated in FIGS. 8A and 8C. FIG. 8C illustrates a hidden line view of malleable retractor 610 showing extending section 630 seated within slot 663 of suction pathway element 660.

In an laterally internal or central direction to slot 633 (that is toward the centerline thereof), suction pathway element 660 also includes one or more extending flexible conduits 664 (illustrated schematically in broken lines in FIG. 8B) which are in fluid connection with connector section 662 at a proximal end thereof and are in fluid connection with ports or opening (slots in the illustrated embodiment) of a suction chamber section 666 at a distal end of suction pathway element 660. During use of malleable retractor 610, matter from outside of malleable retractor 610 is drawn in through slots 667 and into flexible conduit(s) 664 (for example, via one or more ports thereof (not shown) in the vicinity of suction chamber section 666).

In a number of embodiments, malleable body further includes one or more flanges such as flanges 637a and 637b in the vicinity of the proximal end thereof. Flanges such as flanges 637a and 637b may extend in one or more directions and can facility gripping and control of malleable retractor 610 (either manual control or control via one or more other devices as described further below).

In embodiments in which a flexible suction pathway element is used in connection with a malleable body in a malleable retractor hereof, the malleable body (for example, a metal malleable body) provides the malleability required to form the malleable retractor into a desired conformation and maintain that conformation under the forces experienced in a surgical procedure. The flexible suction pathway element provides and efficient pathway via which suction is delivered. As described above, in embodiments in which the flexible suction pathway element is positioned fully interior to the malleable body, the malleable retractor may be readily formed to have two smooth (dorsal/ventral) sides. In that regard, surgeons are accustomed to generally symmetrically formed malleable retractors having two smooth sides so that the instrument may be handed to the surgeon without concern of the orientation of the two sides thereof. Positioning the flexible suction pathway element (fully or partially) on an exterior of a malleable body or a malleable retractor hereof may result in more difficulty in achieving such symmetry. However, positioning the flexible suction pathway element at least partially on an exterior of a malleable body may enable designs which are lighter and/or thinner than designs in which the flexible suction pathway element is positioned on an interior of the malleable body. The use of a malleable body formed, for example, monolithically from a mass of a metal (which may be coated) and used in connection with an external suction pathway element attachable to the malleable body can result in greater ease in conforming the malleable retractor hereof to a desired shape or conformation. The material properties/materials and characteristics of the malleable body and the flexible suction pathway element are readily determined using engineering principles for a given use.

Along with other unique characteristics of the malleable retractors hereof, in a number of embodiments, the body shape and tip topography of the retractors offer distinctive properties. For example, a wide and generally flat body allows significant versatility in the ability of retractors hereof to interface with sensitive tissue (for example, brain tissue). In a number of embodiments (for example, as illustrated in FIGS. 8A through 8C), the tissue interfacing side of the retractor includes a polymeric material (for example, liquid silicone rubber) which may for example, be formed via a surface of the suction pathway element. The distal end of the retractor may include rounded corners. The polymeric material of the suction pathway element may, for example, fully encompass and extend beyond the metal of the malleable body, particularly in regions of the malleable retractor with contact tissue. In other embodiments, the metal of the malleable body may extend beyond the perimeter of the suction pathway element in or more regions of the retractor. Such a polymeric component can mildly compress when retractive forces are applied to the tissue. The exterior surface of the suction pathway element may thus act as a cushion, which mitigates the risk of damage to the tissue's microstructure. Further, the pathway suction elements hereof are low-cost and replaceable, eliminating any challenges associated with sterilization for reuse. Polymeric pathway suction elements are also non-conductive.

A relatively wide body design of malleable retractors hereof results in a relatively larger surface area compared to previously available retractors. The surface area of the retractor distributes the force across the tissue-instrument interface, decreasing the risk of inadvertent tissue damage. The distal suctioning region, chamber or tip (as well as the body of the device) may further include features such as rounded edges and significant width. Further, the functionality of sections of the malleable retractor devices hereof such as the distal end or tip of a number of embodiments of the malleable retractors hereof can be changed. For example, in a number of embodiment the malleable body may be readily removed from connection with the suction pathway element to change characteristics and functionality such as the characteristic of the distal end or tip (including, for example, shape characteristics, material characteristics, suction etc.). For example, different suction holes, hole sizes or bole patterns can be provided depending on the amount of fluid obstructing the view of the surgical field and the rate at which it must be aspirated. Likewise, a non-conductive malleable body (for example, a silicone-coated malleable body) may be used if non-conductive properties are needed. A system or kit hereof may, for example, be provide that include one or more malleable bodies as well as one or more suction path elements to provide control over the properties of the retractor via selection of a particular malleable body and a particular suction path element.

In a number of embodiments hereof in which a suction pathway element hereof forms at least a portion of the exterior surface of at least one side of the retractor, the exposed surface of the suction pathway element my include a generally flat central section and arced or rounded lateral edges. When exposed at the tip or distal end of a retractor hereof, the perimeter shape of the distal end of the suction pathway element may be arced our rounded.

The dimensions of the retractors hereof may vary widely depending on the nature of the use of the retractor. In general, the retractors can vary in length from approximately 7 inches (17.78 cm) to approximately 18 inches (45.72 cm). The retractors can vary in average width from approximately ¼ inch (0.635 cm) to approximately 3 inches (7,62 cm) or more. The width of the retractor can vary over the length thereof. The thickness of retractors hereof is preferably minimized while achieving both retraction and suction. In general, such thickness in no greater that 0.79 inches (2 cm), no greater than 0.59 inches (1.5 cm), no greater than 0.39 inches (1 cm), no greater that 0.30 inches (0.75 cm), or no greater than 0.20 inches (0.5 cm).

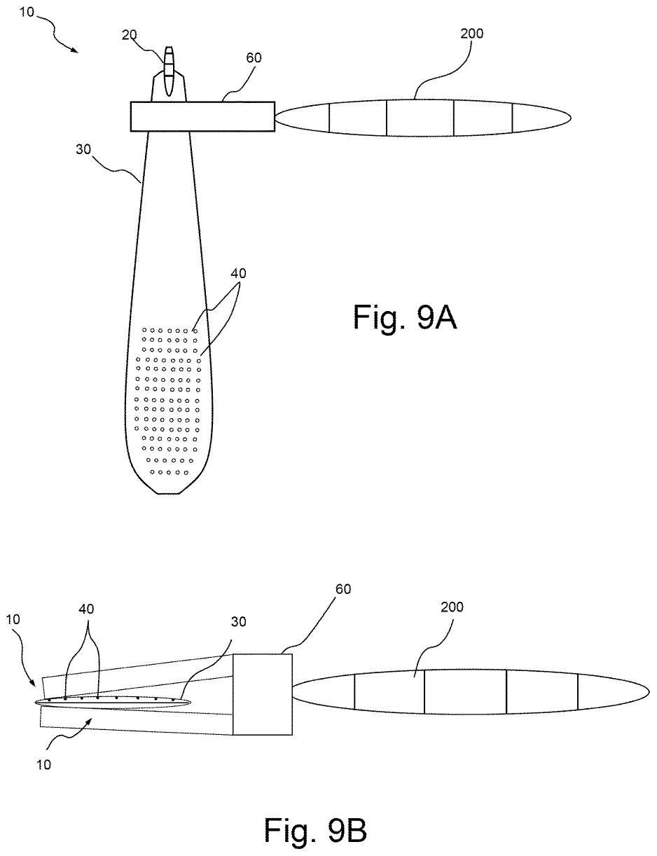

FIG. 9A illustrates a top schematic view of malleable retractor 10 hereof including a section (which may simply be or include a proximal end section or portion of malleable body 30) to cooperate with a connector 60 (for example, a clamp) for use in connection with a retaining system 200, which is configured or operable to maintain the position of malleable retractor 10 (for example, at a fixed position) without manually holding malleable retractor 10. Connector 60 may, for example, be a clamp system of a robotic arm of a robotic control system. FIG. 9B illustrates a side schematic view of a malleable retractor 10 hereof in operative connection with a connector 60 for use in connection with a retaining system 200 (for example, a robotic arm of a robotic control system). An en face view of malleable retractor 10 is provided in the orientation of FIG. 9B wherein malleable retractor 10 is oriented toward the viewer.

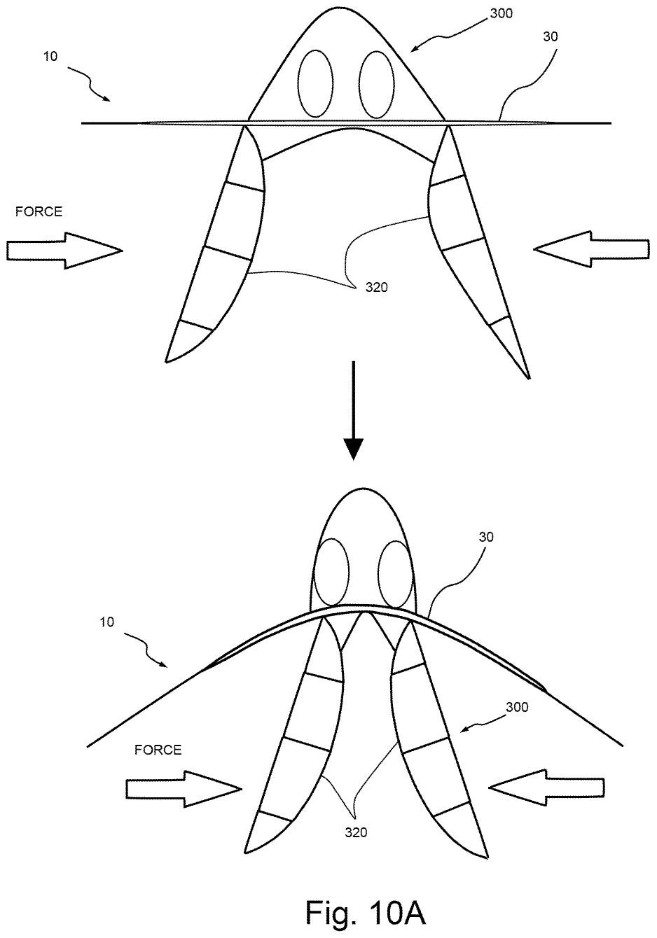

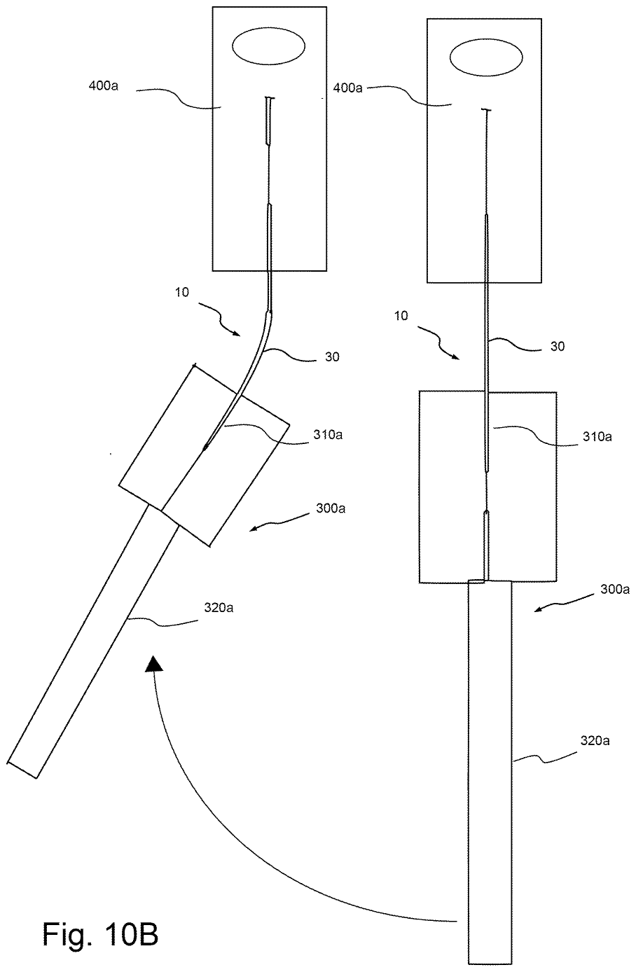

A “bending assist” device or bending tool (for example, as known in the mechanical bending arts), separate from malleable retractor 10 may be desired or required in certain embodiments to assist a surgeon bend a malleable retractor 10 hereof. In that regard, in certain embodiments, a malleable retractor with integrated suction functionality hereof may be more rigid than currently available malleable retractors which do not include an integrated suction functionality, FIG. 10A illustrates a side, schematic view of bending of the malleable body 30 of a malleable retractor 10 hereof using a representative embodiment of a bending tool 300. In the embodiment of FIG. 10A, extending handles 320 of bending tool 300 are squeezed inward to achieve controlled bending of a portion of extending malleable body 30. FIG. 10B illustrates a side, schematic view of bending of the extending malleable body 30 of a malleable retractor 10 hereof with another representative embodiment of a bending tool 300a. In the embodiment of FIG. 10B, a portion of extending malleable body 30 is placed within a slot 310a of bending tool 300a and an extending handle 320a of bending tool 300a is manipulated to achieve the desired bending. An end of body 30 opposite to the end in operative connection with bending tool 300a may be held within a holder 400a. Bending tools such a bending tools 300 and 300a may, for example, provide leverage to multiply and/or focus force applied by a surgeon to effect controlled bending.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A surgical retractor, comprising:
an extending malleable body, the extending malleable body being sufficiently flexible to be bent into a determined conformation for use during a surgical procedure while having sufficient rigidity in the determined conformation to retract soft tissue, and
a suction system in connection with the extending malleable body, the suction system being configured to aspirate material during the surgical procedure, at least a portion of the suction system being configured to conform to the determined conformation of the extending malleable body while maintaining functionality to aspirate the material, the suction system comprising at least one conduit via which the material aspirated from a surgical site can pass through the suction system to be removed from the surgical site, the suction system being configured to be placed in fluid connection with a vacuum source.

2. The surgical retractor of claim 1 wherein the extending malleable body comprises an interior volume in fluid connection with one or more passages formed in a wall of the extending malleable body through which the aspirated material is passable.

3. (canceled)

4. The surgical retractor of claim 1 wherein the extending malleable body comprises an interior volume which forms a part of the at least one conduit.

5. The surgical retractor of claim 2 wherein the suction system comprises a suction pathway element which is flexible and is configured to pass through at least a portion of the internal volume, the flexible suction pathway element comprising at least one conduit and at least one port in fluid connection with the at least one conduit, the at least one port being in fluid connection with the one or more passages formed in a wall of the extending malleable body.

6. The surgical retractor of claim 5 wherein the suction pathway element is formed from a flexible polymeric material.

7. (canceled)

8. The surgical retractor of claim 2 wherein the extending malleable body comprises a first section and the second section that are configured to be placed in an open state and in a closed state, wherein the first section and the second section form the interior volume when in a closed state.

9. The surgical retractor of claim 8 wherein at least one of the first section and the second section comprises the one or more passages.

10. The surgical retractor of claim 9 wherein the suction system comprises a suction pathway element which is flexible and insertable into the internal volume when the first section and the second section are in an open state to pass through at least a portion of the internal volume, the suction pathway element comprising at least one conduit and at least one port in fluid connection with the at least one conduit, the at least one port being in fluid connection with the one or more passages formed in a wall of the extending malleable body,

11. The surgical retractor of claim 10 wherein the suction pathway element forms a sealed engagement with the interior volume to create a suction chamber when the first section and the second section are in a closed state, the suction chamber being in fluid connection with the one or more passages, and the at least one port being positioned within the suction chamber.

12. The surgical retractor of claim 1 wherein the extending malleable body is formed from a malleable metal.

13. (canceled)

14. The surgical retractor of claim 1 wherein the suction system comprises a suction pathway element which is flexible and is configured to be connected to the extending malleable body, the suction pathway element forming at least a portion of the exterior surface of the surgical retractor, the suction pathway element comprising the at least one conduit and at least one port, through which the aspirated material is passable, in fluid connection with the at least one conduit.

15. The surgical retractor of claim 14 wherein the suction pathway element is formed from a flexible polymeric material.

16. (canceled)

17. The surgical retractor of claim 14 wherein the extending malleable body comprises an extending section to which the suction pathway element is attachable.

18. The surgical retractor of claim 17 wherein the suction pathway element comprises a seating into which a portion of the extending malleable body may be positioned to attached the extending section to the suction pathway element.

19. The surgical retractor of claim 18 wherein the extending section comprises a suction chamber positioned on a distal end thereof, the suction chamber comprising an opening on a proximal end thereof via which suction volume positioned on a distal end of the suction pathway element can be passed into the suction chamber, the suction chamber comprising one or more passages therein through which the aspirated material is passable, the at least one port being positioned in the suction volume to be placed in fluid connection with the one or more passages when the distal end of the suction pathway element is within the suction chamber.

20. The surgical retractor of claim 18 wherein the suction pathway element forms at least a portion of an upper surface and a lower surface of the surgical retractor.

21. The surgical retractor of claim 18 wherein the seating extends the full length of the suction pathway element.

22. The surgical retractor of claim 21 wherein the suction pathway element comprises a generally flat upper surface, a generally flat lower surface and a curved surface connecting the generally flat upper surface and the generally flat lower surface.

23. The surgical retractor of claim 21 wherein a distal end of the suction pathway element comprises a rounded edge.

24. The surgical retractor of claim 18 wherein the extending section is U-shaped and the suction pathway element comprises a U-shaped slot around a circumference thereof in which the extending section is seated when the suction pathway element is attached to the extending section.

25. The surgical retractor of claim 24 wherein the suction pathway element comprises a generally flat upper surface, a generally flat lower surface and a curved surface connecting the generally flat upper surface and the generally flat lower surface.

26. The surgical retractor of claim 25 wherein a distal end of the suction pathway element comprises a rounded edge.

27. The surgical retractor of claim 14 wherein the extending malleable body is formed from a malleable metal.

28. (canceled)

29. A method of performing a procedure, comprising: using a retractor during the procedure, the retractor comprising: an extending malleable body, the extending malleable body being sufficiently flexible to be bent into a determined conformation for use during a surgical procedure while having sufficient rigidity in the determined conformation to retract soft tissue, and a suction system in connection with the extending malleable body, the suction system being configured to aspirate material during the surgical procedure, at least a portion of the suction system being configured to conform to the determined conformation of the malleable body while maintaining functionality to aspirate the material, the suction system comprising at least one conduit via which the material aspirated from a surgical site can pass through the suction system to be removed from the surgical site, the suction system being configured to be placed in fluid connection with a vacuum source.

30.-32. (canceled)

33. The method of claim 29 further comprising retaining the retractor in a position during the procedure by operatively connecting the retractor to a retention system.

34. The method of claim 33 wherein the retention system is connected to a robotic control system.

35.-61. (canceled)
